(19) United States
(12) Patent Application Publication
(10) Pub. No.: US 2016/0310734 A1
(43) Pub. Date: Oct. 27, 2016

(54) HANDHELD ELECTRICAL STIMULATION MEDICAL DEVICE

(71) Applicant: MILJØ MOBIL INVEST APS, Rungsted Kyst (DK)

(72) Inventors: Preben Nodskov, Rungsted Kyst (DK); Vibeke Nodskov, Rungsted Kyst (DK); Karin Nodskov Holm, Rungsted Kyst (DK)

(73) Assignee: Xit-International A/S, Horsholm (DK)

(21) Appl. No.: 15/101,916

(22) PCT Filed: Dec. 4, 2014
(86) PCT No.: PCT/DK2014/050413

§ 371 Date: Jun. 5, 2016

(30) Foreign Application Priority Data
Dec. 4, 2013 (DK) BA 2013 00189
Dec. 30, 2013 (DK) BA 2013 00200

(51) Int. Cl. A61N 1/36; A61N 1/04

(52) U.S. Cl. CPC A61N 1/36021; A61N 1/0456; A61N 1/0476; A61N 1/0492

(57) ABSTRACT

The present disclosure relates to a handheld medical device comprising a pen-like exterior casing accommodating: an electrical impulse generator, a holder for an electrical power source, such as a battery, for powering the impulse generator, one or more electrodes incorporated in a proximal end of the casing, a proximal end of said electrode(s) suitable for skin contact and extending from the proximal end of the casing, and a user activated actuator, the device configured such that one or more high-voltage low-current electrical impulses, generated by the impulse generator, is/are transmitted to the electrode(s) upon user activation of the actuator, the device further comprising one or more electrical contact receptacles.

### CROSS-REFERENCE TO RELATED APPLICATIONS

This application claims priority to the PCT Application PCT/DK2014/050413 filed on 04 Dec. 2014 which further claims the priority of Patent applications no BA2013 00189 filed on 04 Dec 2013 filed in the Denmark Patent and Trademark Office which further claims the benefit of Danish Application BA2013 00200 filed on 30 Dec. 2013.The specification of the above referenced patent applications is incorporated herein by reference in its entirety

### FIELD OF INVENTION

The present disclosure relates to a handheld medical device for generating one or more high-voltage low-current electrical impulses through one or more electrodes incorporated in a proximal end of the casing upon user activation.

### BACKGROUND OF INVENTION

Transcutaneous electrical nerve stimulation (TENS or TNS) is the use of electric current produced by a device to stimulate the nerves of a user. TENS covers the complete range of transcutaneously applied currents used for nerve excitation although the term is often used with a more restrictive intent, namely to describe the kind of pulses produced by portable stimulators. A mild sensation is felt by the patient using the TENS system. The pulses block the electrical pain signals going to the brain (Gate control theory of pain). The pulses may also release the body's own natural pain relieving mechanisms (endorphin release). The pulses induce the patient's nervous system to raise its own natural substances, which prevent pain signals from reaching the brain. A TENS device can be used by a patient numerous times throughout the day whenever a patient desires pain relief without risk of overdosing.

A disadvantage of the electric stimulation devices that are available for this purpose is that the electrodes cannot easily be placed optimally on the area that should receive the electrical stimulation. The user may not be capable of reaching the area which needs the electrical stimulation. This may have different causes, for example it could generally be difficult to reach parts of one's own back with a small handheld device, or the user could be incapable of moving his/her body freely. Another problem is that that angle and position of the device may be uncomfortable for the user and inefficient in terms of the electrical stimulation that is delivered. The devices that are described in the art are also inconvenient in terms of activation and deactivation of the device. Furthermore the design of the available device for electrical stimulation is typically somewhat ponderous and not ergonomic. Prior art devices are disclosed in WO 01/01920 and DK 201200147 U3.

### Summary of Invention

The present disclosure relates to a handheld medical device comprising a pen-like exterior casing, accommodating: an electrical impulse generator, a holder for an electrical power source, such as a battery, for powering the impulse generator, one or more electrodes incorporated in a proximal end of the casing, a proximal end of said electrode(s) suitable for skin contact and extending from the proximal end of the casing, and a user activated actuator, wherein the device is configured such that one or more high-voltage low-current electrical impulses, generated by the impulse generator, is/are transmitted to the electrode(s) upon user activation of the actuator.

One purpose of the presently disclosed invention is to increase the flexibility and reach of the handheld device. Therefore, one aspect of the present invention relates to the one or more incorporated electrodes being extendable with a flexible electrode extender and/or an elongated electrode extender comprising an elongated housing accommodation. This can be achieved by designing the one or more incorporated electrodes such that they can be easily connected to the flexible electrode extender or elongated electrode extender. Preferably, the one or more incorporated electrodes form (an) electrical contact receptacle(s). This means that they can be used both in a standalone version of the medical device to transmit electrical impulses directly to the skin of the user, but they can also serve as contact receptacles for an extension of the device. For this purpose the one or more incorporated electrodes are preferably hollow, and/or form female receptacles suitable for connection with a corresponding male plug. Other embodiments of the receptacles are also possible. An extension of the device may be useful in several scenarios. If the user cannot reach certain parts of the body to be exposed to the electrical impulses, an extension of the device may help. This could be for example generally the back of any person or certain specific parts for a disabled person. If for example a telescopic mechanism is incorporated in the elongated housing it is considerably easier for a user to reach some body parts.

It is also possible to have different physical shapes of the electrodes in the extension that transmit electrical impulses to the user depending on the extent of the pain. These shapes of the electrodes in the extension part are easily replaceable if the incorporated electrodes form electrical contact receptacles as described above. The shapes could be for example spherical or pointed, or TENS pads. Furthermore the extensions can be split into several electrodes. The advantage of splitting the extensions is that several areas on the body can be exposed to electrical impulses simultaneously. A further aspect of the presently disclosed invention relates to the medical device having two electrodes extending from the proximal end of the casing to transfer the electrical impulses. The two electrodes may generate a current flow during the electrical pulse between the two electrodes. When using two electrodes extending from the proximal end of the casing, one of them can act as ground and therefore it is no longer needed to touch a part of the grip of the device, which typically serves as ground in the known devices.

### DESCRIPTION OF DRAWINGS

FIG. 1a shows a schematic drawing of a handheld medical device (100) with a pen-like exterior casing (101) configured to generate one or more high-voltage low-current electrical impulses through two electrodes upon user activation.

FIG. 1b shows a cross section of the handheld medical device (100) of FIG. 1b.

FIG. 2a shows a handheld medical device (200) with a pen-like exterior casing (201) having an electrical contact receptacle (300) configured to be releasably attached to the proximal end of the exterior casing. The figure also shows a male plug (210), which can be connected to the receptacle (300).

FIG. 2b shows a handheld medical device (400) with a pen-like exterior casing (401) configured to generate one or more high-voltage low-current electrical impulses through one electrode (405), which also forms an integrated electrical contact receptacle (410) (hollow female socket with an annulus (406) at the proximal end of the external casing).

FIG. 3a shows a handheld medical device (500) with a pen-like exterior casing (501) configured to generate one or more high-voltage low-current electrical impulses through one electrode (505), which also forms an integrated electrical contact receptacle (540). The handheld device further comprises an electrical extension cable (520), an elongated electrode extender (510) (having a telescopic mechanism incorporated in the elongated housing), and a Y splitter cable (530) with two electrode assemblies (531) suitable for skin contact.

FIG. 3b shows an alternative version of the elongated electrode extender (510) with a telescopic mechanism incorporated in the elongated housing, comprising a spherical metallic electrode (511) suitable for skin contact. FIG. 4a shows the handheld medical device (100) of FIG. 1.

FIG. 4b shows the handheld activation of the medical device (100) of FIG. 4a.

FIG. 5a shows the handheld activation of the medical device (100) of FIG. 2a to which a flexible electrode extender in the form of an electrical extension cable and an electrode assembly (531) has been connected.

FIG. 5b shows two adhesive electrode assemblies (531) releasably and adhesively attached to the back of a user.

FIG. 5c shows two adhesive electrode assemblies (531) releasably and adhesively attached to the neck of a user.

### DETAILED DESCRIPTION OF THE INVENTION

The present disclosure relates to a handheld medical device comprising a pen-like exterior casing, accommodating: an electrical impulse generator, a holder for an electrical power source, such as a battery, for powering the impulse generator, one or more electrodes incorporated in a proximal end of the casing, a proximal end of said electrode(s) suitable for skin contact and extending from the proximal end of the casing, and a user activated actuator, wherein the device is configured such that one or more high-voltage low-current electrical impulses, generated by the impulse generator, is/are transmitted to the electrode(s) upon user activation of the actuator. In one embodiment of the present invention the one or more high-voltage low-current electrical impulses is/are in the form of transcutaneous nerve stimulation (TNS) and/or transcutaneous electrical nerve stimulation (TENS).

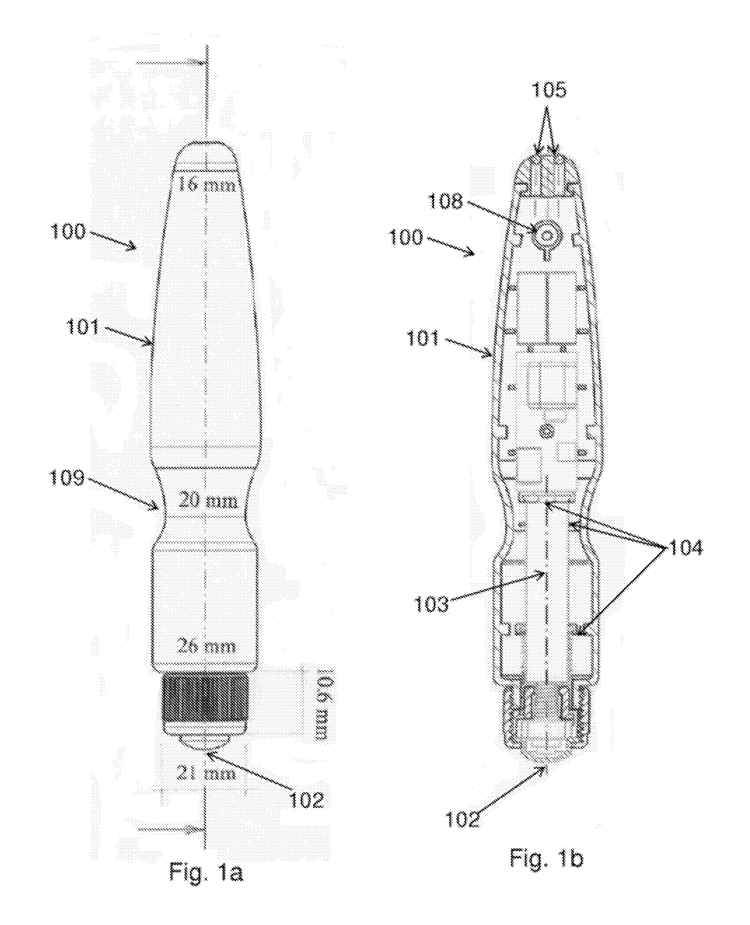

One purpose of the presently disclosed invention is to make it possible to use the device without having to touch the grip of the device, which serves as electrical ground in the devices that are available on the market. One aspect of the presently disclosed invention therefore relates to the medical device having two electrodes extending from the proximal end of the casing to transfer the electrical impulses. FIG. 1b shows an example of a handheld medical device with a pen-like exterior casing configured to generate one or more high-voltage low-current electrical impulses through two electrodes (105) upon user activation. The two electrodes may generate a current flow during the electrical pulse between the two electrodes (from one electrode to the other). In the devices known in the art there is only one electrode, at which a higher potential is generated during the electrical impulse; a part of the grip of the device typically serves as electrical ground, which means that the user needs to hold/touch the grip in to use the device. When using two electrodes extending from the proximal end of the casing according to the present invention, one of them can act as ground (more generally there can be a difference in electrical potential between the two electrodes). With this configuration there is no need for the user to touch the grip (ground) of the device, which simplifies the use. The present invention preferably has an electrically insulating exterior casing. An electronics module in combination with a high voltage transformer can be configured to generate electrical impulses suitable for TNS/TENS. The voltage of the impulses are around 10.000-20.000, typically 15-17.000 kV with a current of between 0,000001 to 0,00001 A, typically around 0,000006 A. Examples of electronics and high voltage transformer are disclosed in WO 01 /01920 and DK 201200147 U3.

A further aspect of the present invention relates to increasing the flexibility and reach of the handheld device. The inventors have realized that the one or more electrodes, which is/are incorporated in a proximal end of the casing with a proximal end of said electrode(s) suitable for skin contact and extending from the proximal end of the casing, could also act as receptacles, i.e. being able to establish an electrical connection with an additional connector. The advantage of this solution is that the device can be used both as a small standalone handheld device for relieving pain, i.e. it can easily be carried in e.g. a purse or small bag and is very convenient to use in most situations; however, since the electrode(s) is/are also capable of connection with additional connectors, the device can be extended to reach parts of the body which is otherwise difficult to reach, e.g. the back of the user. Even though the shape of the medical device in its original form is convenient for most positions, there could also be use cases where the physical design of the medical device is not ideal. By attaching extension(s) and/or differently shaped electrodes to the device it is possible to achieve a more efficient use of the device for a number of scenarios or situations.

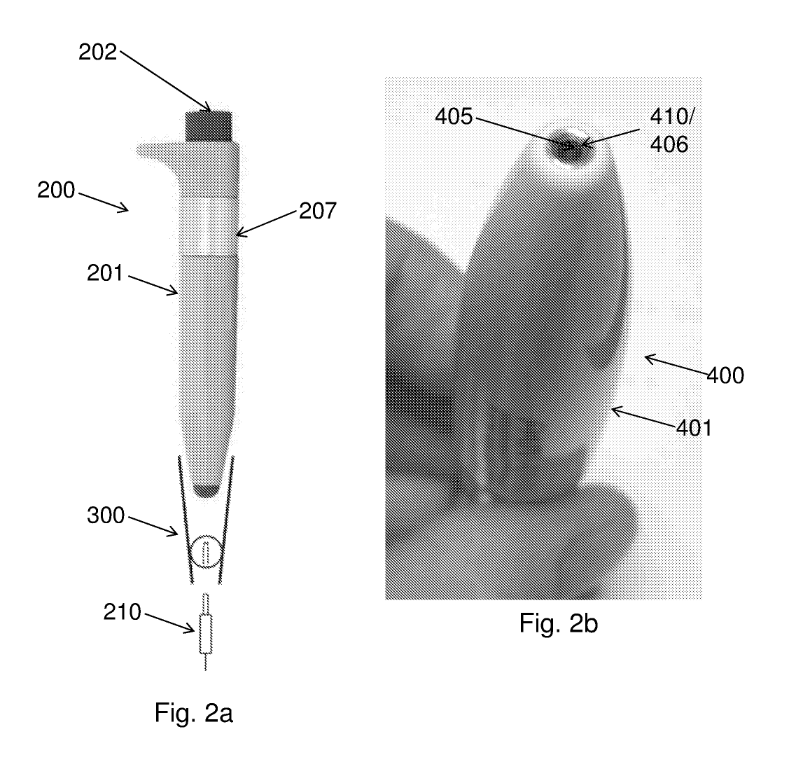

In one embodiment of the present invention the electrode(s) are hollow. In one embodiment the electrodes can be described as elongated hollow sockets extending longitudinally into the proximal end of the external casing. FIG. 2b shows an example of a handheld medical device (400) with a pen-like exterior casing (401) configured to generate one or more high-voltage low-current electrical impulses through one electrode (405), which also forms an integrated electrical contact receptacle (410). In this example the electrode is a hollow female socket, which can serve both as an electrode for transferring the electrical impulses to the skin of the user, or as an integrated electrical contact receptacle being used to extend or improve the device. In the example above the electrode comprises an annulus (406) at the proximal end of the external casing forming the contact area to the user. In one embodiment of the present invention the diameter of the annulus is between 2 and 6 mm, such as approx. 4 mm, and the width of the annulus is between 1 and 3 mm, such as approx. 2 mm. In one embodiment the electrode protrudes less than 1 mm from the proximal end of the casing, or less than 0.8 mm, or less than 0.6 mm, such as approx. 5 mm, or at least 0.1 mm, or at least 0.2 mm, or protruding at least 0.4 mm from the proximal end of the casing. The electrode is typically metallic, and can be made of for example gold, silver, nickel, copper, zinc or an alloy hereof.

In one embodiment of the present embodiment the inside contact diameter of the hollow electrodes and/or the female plug is less than 2.5 mm, or less than or equal to 2 mm, or less than 1.5 mm, preferably less than or equal to 1 mm. The inside contact surface of each hollow electrode and/or female plug may be adapted to establish an electrical connection with a male connector with an external diameter of less than 2.5 mm, or less than or equal to 2 mm, or less than 1.5 mm or less than or equal to 1 mm.

In another embodiment of the presently disclosed invention the electrical contact receptacle(s) is/are configured to be releasably attached to the proximal end of the exterior casing. This gives further freedom to design the electrodes for improved use in a standalone version of the medical device, and still maintain the possibility to extend the reach of the electrodes by first adding a releasably attached electrical contact receptacle and extensions. FIG. 2a shows such an electrical contact receptacle (300) being configured to be attached to the proximal end of the exterior casing. The figure also shows a male plug (210), which can be connected to the receptacle. In one embodiment the electrical contact receptacle is formed as a cap and configured to be snap-fitted to and cover the proximal end of the exterior casing. In the electric stimulation devices that are available, the user typically has to push an activation button to generate one electrical impulse. In the present invention the device is configured such that the generation of the one or more high-voltage low-current electrical impulses can be provided upon user activation of the actuator and such that the generation of the one or more high-voltage low-current electrical impulses continues until the generation of electrical impulses is terminated by additional user activation (deactivation) of the actuator. This is an improvement of the use of the device, in particular for persons having difficulties to push the activation button repeatedly (e.g. arthritis patients, elderly or sick). One embodiment of the present invention relates to a timer. A specific time during which electrical impulses should be generated, is set, and when the device is activating the timer counts down and switches itself off when the timer has reach zero. Since the user may have specific needs for pain relief, the one or more high-voltage low-current electrical impulses is/are preferable predefined with regard to one or more of the following parameters: number of electrical impulses, duration of each impulse, voltage and current of each impulse, duration between impulses, total duration of the high-voltage low-current electrical impulses. In one embodiment it is therefore possible to program the device with regard to the recited parameters and activate it accordingly by activating the actuator.

The present invention also relates to further improvements for the handling and use of the electric stimulation device. By having a rechargeable power source, preferably configured such that the power source can be recharged via an electrical connection through the electrode(s), the user does not have to purchase new batteries. The concept of having a rechargeable power source, which can be recharged via an electrical connection through the electrode(s), also means that the physical design of the device becomes simpler since no mechanical solutions (battery door etc.) are needed for replacing the battery, which also makes a slimmer design possible.

Furthermore a rechargeable power source can be made to have less environmental impact than a disposable battery.

The medical device may further comprise a flexible electrode extender comprising one or more electrode assemblies suitable for skin contact, the electrode assemblies connected via a cable to a plug configured for forming an electrical connection with the electrical contact receptacle of the handheld medical device, such that the one or more high-voltage low-current electrical impulses provided from the medical device upon user activation of the actuator is/are provided via said one or more electrode assemblies. The inventors have realized that this solution enables greater flexibility and reach of the device. One example of a scenario, in which it would be beneficial to have this reach and flexibility, is for pain in the back regions of the user. It is difficult for the user to reach the back with the handheld device. Using the feature of a handheld device further comprising a flexible electrode extender as described, and, for example placing self-adhering pads (electrode assemblies) connected through the flexible extender, enables the user to relieve pain in a wide range of scenarios, and still enjoy the advantages and simplicity of a handheld device.

In one embodiment the cable is a splitter configured to split the one or more high-voltage low-current electrical impulses from the medical device to at least two electrode assemblies. This also gives the user the possibility to relieve the pain in several areas simultaneously, still while maintaining the advantages of a handheld device. In one embodiment the plug is a male plug, such as a banana plug, suitable for connection with a corresponding female receptacle of the medical device. This is a simple and reliable solution, which is in line with the convenience of the idea of a handheld electric stimulation device for relieving pain. The outside contact diameter of the male plug may be less than 2 mm, or less than 1.5 mm, preferably less than or equal to 1 mm.

The one or more electrode assemblies suitable for skin contact could take several different shapes and sizes. In one embodiment at least one of said electrode assemblies is formed as an adhesive patch configured to be releasably and repeatably adhesively attached to the skin of a user. TENS pads come in a wide range of sizes, shapes and material types. In another embodiment at least one of the electrode assemblies comprises metallic electrodes adapted for contacting the skin of a user. The metallic electrodes could have the same shape as the handheld device or another specific shape such as spherical or pointed.

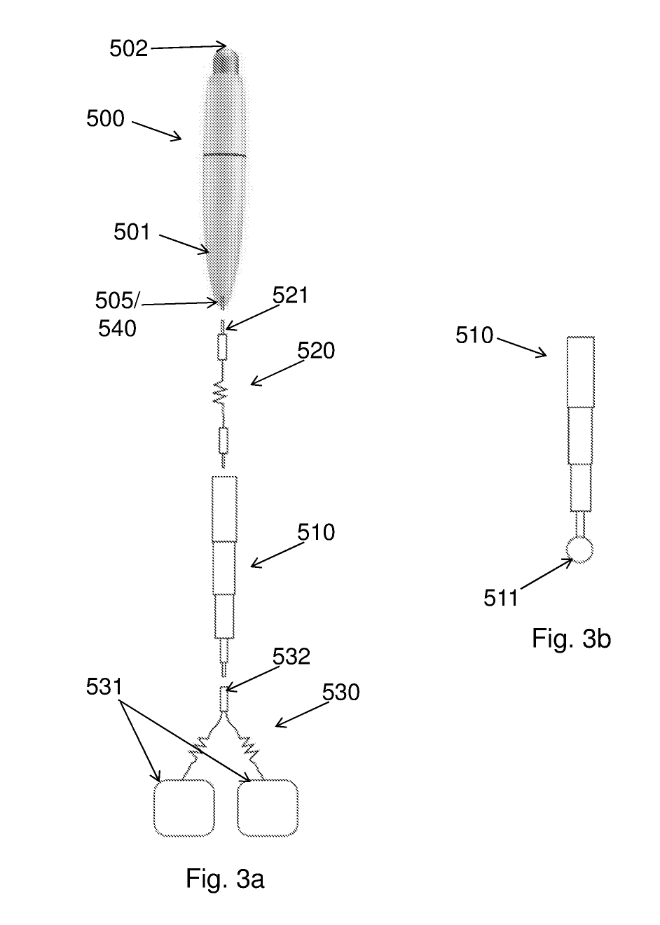

The length of the cable is preferably in a range that makes it possible to reach all body parts of a user, or the part that is intended to be exposed to the electric stimulation and cannot be reached with the handheld device, such as the back of the user, from a position where the handheld device is placed in the hand of the user. In one embodiment of the invention the length of the cable is at least 20 cm, or at least 30 cm, or at least 40 cm, or at least 50 cm, or at 60 cm, or at least 80 cm, or at least 100 cm. The present invention also relates to the medical device further comprising an elongated electrode extender comprising an elongated housing accommodating one or more elongated electrodes suitable for skin contact at a proximal end and a handle at a distal end, and configured to be electrically connected to the electrical contact receptacle of the handheld medical device, such that the one or more high-voltage low-current electrical impulses provided from the medical device upon user activation of the actuator is/are provided via said one or more elongated electrodes. Preferably the elongated electrode extender is substantially rigid. The inventors have realized that by having a substantially rigid, elongated electrode extender comprising an elongated housing accommodating, the position of the electrode(s) which provide the electrical impulses to the skin of the user can be actively adjusted directly by the user holding the extender in his/her hand. Since the electrodes are situated at a certain distance from the point of attachment, the reach of the device is longer than when it is used as a standalone device. Preferably the electrodes have metallic ends adapted for contacting the skin of a user. The metallic electrodes could have the same shape as the handheld device or another specific shape such as spherical, e.g. in the form of a metallic ball-shaped electrode, or pointed. The medical device may also further comprising one or more Y splitter cables (531 in the example shown in FIG. 3a), and one or more electrode assemblies suitable for skin contact, each electrode assembly connected via a cable to the elongated electrode extender. Furthermore the elongated electrode extender may be connected to the handheld medical device with an electrical extension cable (520 in the example shown in FIG. 3a).

A telescopic mechanism may be incorporated in the elongated housing of the electrode extender such that the length of the elongated electrode extender can be varied. The telescopic mechanism makes it possible to increase or decrease the length to a position which is suitable for use on a specific body part holding the device in a specific position.

### EXAMPLES

The invention will in the following be described in greater detail with reference to the drawings. The drawings are exemplary and are intended to illustrate some of the features of the present method and unit and are not to be construed as limiting to the presently disclosed invention.

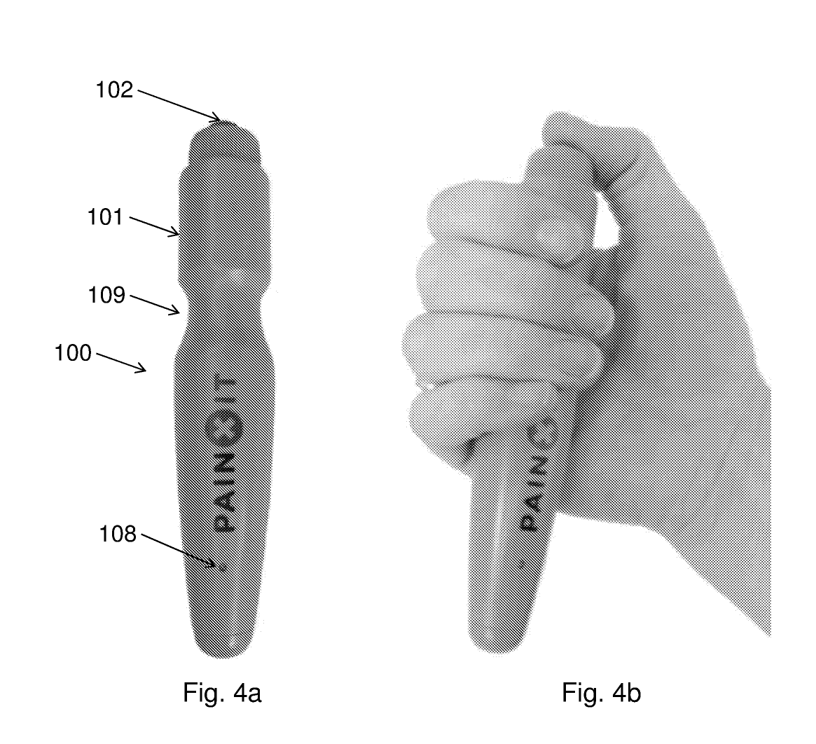

FIGS. 1 a and 4a show a handheld medical device (100) with a pen-like exterior casing (101) configured to generate one or more high-voltage low-current electrical impulses by means of two electrodes upon user activation. The device can be activated/deactivated by means of an activator (actuator) (102). FIG. 1b shows a cross section of the handheld medical device (100) of FIG. 1b. In addition to the items shown in FIG. 1a an internal battery (103) and battery holder (104) are visible. Furthermore two electrodes (105), incorporated in a proximal end of the casing (101), configured to transfer one or more high-voltage low-current electrical impulses, are shown. An impulse indicator (108) located at the side of the casing (102) flashes concurrently with the provision of the electrical impulse for a user to visibly follow when an impulse is provided to the electrodes. The casing (101) is provided with a circumferential recess (109) (of FIGS. 1 and 4) for improved ergonomics as illustrated in FIG. 4b showing the hand of a user holding the medical device (100) of FIG. 4a with the thumb operating the actuator (102).

FIG. 2a shows a handheld medical device (200) with a pen-like exterior casing (201) having an electrical contact receptacle (300) configured to be releasably attached to the proximal end of the exterior casing. The figure also shows a male plug (210), which can be connected to the receptacle (300). Furthermore a grip (207), typically serving as electrical ground in previous solutions, is shown. In this regard the shown example does not include the solution having two electrodes, wherein one acts as ground, as proposed in the present invention.

FIG. 2b shows a handheld medical device (400) with a pen-like exterior casing (401) configured to generate one or more high-voltage low-current electrical impulses through one electrode (405), which also forms an integrated electrical contact receptacle (410) (in form of a hollow female socket with an annulus (406) at the proximal end of the external casing). In this example the electrode (405) is an integrated part of the electrical contact receptacle (410).

FIG. 3a shows a handheld medical device (500) with a pen-like exterior casing (501) configured to generate one or more high-voltage low-current electrical impulses through one electrode (505), which also forms an integrated electrical contact receptacle (540). The handheld device further comprises an electrical extension cable (520) with a male connector (521), an elongated electrode extender (510) (having a telescopic mechanism incorporated in the elongated housing), and a Y splitter cable (530) with two electrode assemblies (531) suitable for skin contact, and a.female connector (532). FIG. 3b shows an alternative version of the elongated electrode extender (510) with a telescopic mechanism incorporated in the elongated housing, comprising a spherical metallic electrode (511) suitable for skin contact.

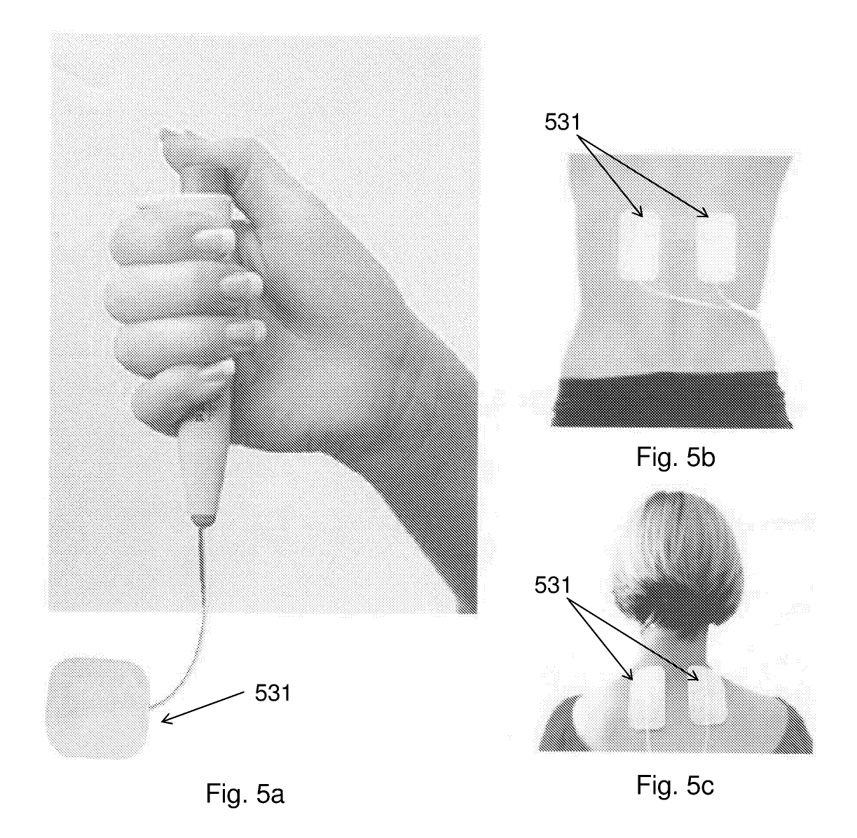

FIG. 5a shows the principle of a handheld activation of the medical device (200) of FIG. 2a to which a flexible electrode extender in the form of an electrical extension cable and an electrode assembly (531) have been connected. This figure is for illustrative purpose with the rather short extension cable. FIGS. 5b and 5c show two examples of the provision of pairs of electrode assemblies adhesively attached to the body of the user at locations (back and neck) that would be difficult to reach by means of the handheld device itself. With these flexible electrode extenders the user can still hold and operate the medical device in one hand but the TENS stimulation can be provided anywhere on the body.

1. A handheld medical device comprising
a pen-like exterior casing accommodating:
an electrical impulse generator,
a holder for an electrical power source, such as a battery, for powering the impulse generator,
one or more electrodes incorporated in a proximal end of the casing, a proximal end of said electrode(s) suitable for skin contact and extending from the proximal end of the casing, and
a user activated actuator,
the device configured such that one or more high-voltage low-current electrical impulses, generated by the impulse generator, is/are transmitted to the electrode(s) upon user activation of the actuator, the device further comprising one or more electrical contact receptacles.

2. The medical device according to claim 1, comprising two electrodes wherein the electrical impulses is transmitted between the electrodes upon user activation of the actuator.

3. The medical device according to any of the preceding claims, wherein said electrode(s) are hollow.

4. The medical device according to any of the preceding claims, wherein the electrical contact receptacle(s) is/are configured to be releasably attached to the proximal end of the exterior casing.

5. The medical device according to claim 4, wherein the electrical contact receptacle is formed as a cap and configured to be snap-fitted to and cover the proximal end of the exterior casing.

6. The medical device according to any of preceding claims, wherein the electrode(s) are an integrated part of the electrical contact receptacle, the electrical contact receptacle being incorporated in the proximal end of the casing.

7. The medical device according to any of the preceding claims 4-6, wherein the electrical contact receptacle forms a female receptacle suitable for connection with a corresponding male plug.

8. The medical device according to any of the preceding claims 7, wherein each electrode comprises an elongated (hollow) socket extending longitudinally into the proximal end of the external casing, each electrode thereby forming part of the female receptacle.

9. The medical device according to any of the preceding claims 8, wherein each electrode comprises an annulus at the proximal end of the external casing forming the contact area to the user.

10. The medical device according to any of the preceding claims 9, wherein the diameter of the annulus is between 2 and 6 mm, such as approx. 4 mm, and wherein the width of the annulus is between 1 and 3 mm, such as approx. 2 mm.

11. The medical device according to any of the preceding claims, wherein each electrode protrudes less than 1 mm from the proximal end of the casing, or less than 0.8 mm, or less than 0.6 mm, such as approx. 5 mm, or at least 0.1 mm, or at least 0.2 mm, or protruding at least 0.4 mm from the proximal end of the casing.

12. The medical device according to any of the preceding claims, wherein the electrode(s) are metallic.

13. The medical device according to any of the preceding claims, wherein the device is configured such that upon user activation of the actuator the one or more high-voltage low-current electrical impulses is/are predefined with regard to one or more of the following parameters: number of electrical impulses, duration of each impulse, voltage and current of each impulse, duration between impulses, total duration of the high-voltage low-current electrical impulses.

14. The medical device according to any of the preceding claims, wherein the device is configured such that the generation of the one or more high-voltage low-current electrical impulses can be provided upon user activation of the actuator and such that the generation of the one or more high-voltage low-current electrical impulses can be terminated by additional user activation (deactivation) of the actuator.

15. The medical device according to any of the preceding claims, wherein the power source is rechargeable.

16. The medical device according to any of the preceding claims 15, wherein the device is configured such that the power source can be recharged via an electrical connection through the electrode(s).

17. The medical device according to any of the preceding claims, wherein the inside contact diameter of the hollow electrodes and/or the female plug is less than 2.5 mm, or less than or equal to 2 mm, or less than 1.5 mm, preferably less than or equal to 1 mm.

18. The medical device according to any of the preceding claims, wherein the inside contact surface of each hollow electrode and/or female plug is adapted to establish an electrical connection with a male connector with an external diameter of less than 2.5 mm, or less than or equal to 2 mm, or less than 1.5 mm or less than or equal to 1 mm.

19. The medical device according to any of the preceding claims, wherein a telescopic mechanism is incorporated in the exterior casing such that the length of the device can be extended.

20. The medical device according to any of the preceding claims, wherein the one or more high-voltage low-current electrical impulses is/are suitable for transcutaneous nerve stimulation (TNS) and/or transcutaneous electrical nerve stimulation (TENS) of a user.

21. The medical device according to any of the preceding claims, further comprising a flexible electrode extender comprising one or more electrode assemblies suitable for skin contact, the electrode assemblies connected via a cable to a plug configured for forming an electrical connection with the electrical contact receptacle of the handheld medical device, such that the one or more high-voltage low-current electrical impulses provided from the medical device upon user activation of the actuator is/are provided via said one or more electrode assemblies.

22. The medical device according to claim 21, wherein the cable is a splitter configured to split the one or more high-voltage low-current electrical impulses from the medical device to at least two electrode assemblies.

23. The medical device according to any of preceding claims 21-22, wherein the plug is a male plug, such as a banana plug, suitable for connection with a corresponding female receptacle of the medical device.

24. The medical device according to claim 23, wherein the outside contact diameter of the male plug is less than 2 mm, or less than 1.5 mm, preferably less than or equal to 1 mm.

25. The medical device according to any of preceding claims 21-24, wherein at least one of said electrode assemblies is formed as an adhesive patch configured to be releasably and repeatably adhesively attached to the skin of a user.

26. The medical device according to any of preceding claims 21-25, wherein at least one of said electrode assemblies comprises two metallic electrodes adapted for contacting the skin of a user when said assembly is attached to said skin.

27. The medical device according to any of preceding claims 21-26, wherein the plug of the flexible electrode extender is configured such that one or more of the flexible electrode extenders can be connected to the handheld medical device.

28. The medical device according to any of preceding claims 21-27, wherein the length of the cable is at least 20 cm, or at least 30 cm, or at least 40 cm, or at least 50 cm, or at 60 cm, or at least 80 cm, or at least 100 cm.

29. The medical device according to any of the preceding claims, further comprising an elongated electrode extender comprising an elongated housing accommodating one or more elongated electrodes suitable for skin contact at a proximal end and a handle at a distal end, and configured to be electrically connected to the electrical contact receptacle of the handheld medical device, such that the one or more high-voltage low-current electrical impulses provided from the medical device upon user activation of the actuator is/are provided via said one or more elongated electrodes.

30. The medical device according to claim 29, wherein the elongated electrode extender is substantially rigid.

31. The medical device according to any of preceding claims 29-30, wherein the elongated electrode extender is connected to the handheld medical device with an electrical extension cable.

32. The medical device according to any of preceding claims 29-31, comprising one spherical metallic electrode suitable for skin contact.

33. The medical device according to any of preceding claims 29-32, wherein a telescopic mechanism is incorporated in the elongated housing such that the length of the elongated electrode extender can be varied.

34. The medical device according to any of preceding claims 29-33, further comprising one or more Y splitter cables, and one or more electrode assemblies suitable for skin contact, each electrode assembly connected via a cable to the elongated electrode extender.
